(19) United States
(12) Patent Application Publication
(10) Pub. No.: US 2024/0293613 A1
(43) Pub. Date: Sep. 5, 2024

(54) PIEZO-ACTUATED DRIVE UNIT FOR SYRINGE PUMP

(71) Applicant: B. Braun Melsungen AG, Melsungen (DE)

(72) Inventor: Marcel Stich, Nieste (DE)

(21) Appl. No.: 18/571,230

(22) PCT Filed: Jun. 14, 2022
(86) PCT No.: PCT/EP2022/066121

§ 371 Date: Dec. 17, 2023

(30) Foreign Application Priority Data
Jun. 18, 2021 (DE) 10 2021 115 809.5

(51) Int. Cl. A61M 5/145; A61M 5/142; A61M 5/172

(52) U.S. Cl. CPC A61M 5/14566; A61M 5/172; A61M 2005/14208

(57) ABSTRACT

A piezoelectric drive unit for a syringe pump includes a drive frame, a control unit, a push rod movable on the drive frame in an oscillating manner via a longitudinal stroke device with a first piezo actuator, a linear guide, and a carriage traversable along the linear guide. A first clamping device is coordinated by the control unit for alternating coupling and decoupling of the carriage to or from the push rod. During coupling, an advance of the carriage is generated according to the longitudinal stroke of the push rod movable from an axial starting position. In the first decoupling state, the push rod is released for a motion separate from the carriage. The first clamping device includes a second piezo actuator connected to the control unit. The second piezo actuator is configured to cause at least the first coupling state by expansion or contraction thereof.

### CROSS-REFERENCE TO RELATED APPLICATIONS

This application is the United States national stage entry of International Application No. PCT/EP2022/066121, filed on Jun. 14, 2022, and claims priority to German Application No. 10 2021 115 809.5, filed on Jun. 18, 2021. The contents of International Application No. PCT/EP2022/066121 and German Application No. 10 2021 115 809.5 are incorporated by reference herein in their entireties.

### FIELD

The present disclosure relates to a piezoelectric drive unit for a medical-technical use, especially for installation in a medical-technical syringe pump, infusion pump, dosing pump and the like. Furthermore, a corresponding method for dispensing and/or dosing of medical liquids is disclosed. In addition, a corresponding medical-technical syringe pump with the piezoelectric drive unit and a component corresponding thereto are proposed. Further, a computer program on a data medium for executing the method by a control unit of the piezoelectric drive unit according to the disclosure is proposed.

### BACKGROUND

In the medical technology, syringe pumps or, respectively, infusion pumps are frequently used for precise conveying or, respectively, dosing of medical liquids such as medications, active ingredient solutions, infusions and the like supplied or, respectively, prepared in liquid form. Thereby, the conveying or, respectively, dosing is especially carried out by controlled squeezing of a syringe containing the active ingredient solution, inserted into the syringe pump. An example of a syringe pump on the market is known under the trade name “Space plus Perfusor” from the company B. Braun. For precise conveying or, respectively, dosing, a drive unit installed in the syringe pump causes a linear movement of a corresponding syringe piston. This linear movement for conveying or, respectively, dosing is usually achieved by means of spindle drives as well as reduction gears for advancing a piston plunger of the drive unit acting on the syringe piston. However, with regard to the design of such drive systems, it is disadvantageous that the number of moving parts to be fitted to each other is high, which causes increased wear points and sources of error. As a result, important aspects of such systems are disadvantageously limited, especially with regard to their accuracy, durability, robustness, maintenance requirements and manufacturing costs.

Furthermore, in the field of drive technology for metering or, respectively, precision pumps, the use of piezoelectric elements (units) or, respectively, piezo actuators (piezo actuators; in short: piezos) is known in principle, especially for the formation of piezoelectric servo drives, but also otherwise. For example, WO 2016/153171 A1 relates to a dispensing pump in a method for manufacturing of electronics with a linear drive for direct valve metering of a liquid such as a synthetic resin, which is metered directly from a feed reservoir in a valve body via a nozzle by means of actuation of a valve rod. Thereby, by means of the periodic expansion and linear deformation of a piezoelectric unit, a guide rod of the linear drive, along which a movable element arranged on the valve rod can move, is moved linearly for direct valve metering.

Furthermore, in JP 2006 281178 A an especially miniaturizable syringe pump for a manufacturing line in the semiconductor manufacturing is disclosed, which is capable to discharge a minute amount, e.g. 0.1 microliter, of even a viscous liquid, such as of an adhesive or of a paste, at high speed with high precision. For this purpose, due to the expansion and contraction of a piezoelectric element, a movable device of a piston provided with a drive rod can be moved precisely by a minute amount in advance direction. When the drive rod thereby moves in a first direction for advancing, a rotatably supported ball of a coupling element is brought into pressure contact for its locking. Thereby, the locking causes that the motion of the coupling element is locked when the drive rod moves in a second direction. When using such a syringe pump attached to a robot arm, its motions can be realized with a high speed as well as acceleration, which shortens the cycle time of the manufacturing line. Insofar as the solution proposed herein comprises the coupling element with the ball rotatably supported on an inclined surface as well as slider and spring elements, it is nevertheless clearance-affected in a disadvantageous manner or, respectively, requires a complex interaction of individual fits of the components to each other. It is additionally disadvantageous with regard to wear problems that very high, namely point-like acting, forces or, respectively, load peaks of the surface pressure (cf. Hertzian pressure with contact of curved surfaces) occur in the coupling element with the pressure contact of the rotatably supported ball which causes the locking. Furthermore, due to the small stroke or, respectively, the small metering quantity, an operable conveying rate is correspondingly low.

Further, CN 102192135 discloses a piezoelectric pump which is based on the basic principle of a positive displacement pump in modification of a membrane pump known as such for the medical technology. Thereby, the stroke movement of the membrane is not generated in a usual way by means of a rotary motor, but by means of a piezo.

Again, from the EP 2198164 B1 as well as the EP 3337534 A1 medical-technical syringe pumps (drives) are known in which piezoelectricity is used for generation of rotation or, respectively, a torque in an otherwise conventional uni-directional spindle drive (as described at the beginning). For example, EP 3337534 A1 discloses a drive mechanism for a portable piezoelectric (microfluid) pump for drug delivery from a syringe, provided in a cartridge, to a patient. In the pump, piezoelectric elements, when energized, for this purpose, cause a linear motion of a push rod, which is in connection to the syringe, to drive the syringe forward and to deliver the drug. Thereby, the high torque which is generated by the piezoelectric elements is directly converted into the same torque at the lead screw, so that no torque-increasing reduction system is required.

In the aforementioned state of the art, there are thus a number of disadvantages or, respectively, design-related limitations. Especially, with medical-technical syringe pumps in the design with (piezoelectric) spindle drive, there is (still) the problem that these are clearance-affected. In this respect, spindle drives require many intermeshing, moving components (spindle, gears, motor, bearing, etc.). In order to achieve a desired or, respectively, required accuracy or, respectively, precision of conveying or, respectively, dosing of the medical liquid in this design, very tight dimensional tolerances or, respectively, fits must be selected. Thereby, the individual dimensional tolerances or, respectively, fits must be coordinated with each other in the design and correspondingly verified or, respectively, ensured in the quality control after their manufacture. Furthermore, moving components are subject to wear; as a result, this brings a medium-term loss of accuracy or, respectively, costly maintenance requirements.

Furthermore, the above-mentioned design with (piezoelectric) spindle drive disadvantageously requires an increased space requirement, which exists despite a small stroke or, respectively, despite a correspondingly low conveying rate (or, respectively, pumping capacity). In addition, further disadvantages of increased energy consumption, of the generation of waste heat or, respectively, of operation-related noise become apparent during the operation. Furthermore, in the case of stride drives and oscillating drives, a low holding force is to be noted as disadvantage. Consequently, the conventional design offers only a limited suitability for use in mobile patient devices such as portable infusion pumps.

Thus, there is a need to improve the above mentioned aspects and to overcome the disadvantages in the prior art. One object which is to be solved by the present disclosure is to provide a quieter, more accurate and more stable drive unit for use in medical-technical dosing or, respectively, precision pumps, especially for infusion pumps. Thereby, adjustment and calibration efforts are to reduced. Especially, the drive unit, when used in a medical-technical syringe pump, should be suitable to provide a uniform, precise dosing, especially drug delivery, over longer periods of time. Further, it is an objective to provide a design for a drive unit that shows a lower or even no susceptibility to wear, despite high forces. Other objects are to design the drive unit and thus the entire pump lighter as well as to increase the energy efficiency.

According to the disclosure (or, respectively, according to a first-mentioned aspect of the present disclosure), a piezoelectric drive unit comprises (or, respectively, has): a drive frame, a control unit, a push rod, a linear guide, a carriage and a first clamping device of the carriage. Thereby, the piezoelectric drive unit is configured for dispensing and/or dosing of medical liquids from a medical container. Especially, the piezoelectric drive unit is configured for (especially for installation in) a medical-technical syringe pump, infusion pump or the like travelling in an axial longitudinal direction. Thereby, the push rod is configured at the drive frame to be movable in an oscillating manner about a longitudinal stroke in the longitudinal direction by means of a longitudinal stroke device, in order to be moved back and forth with acting of at least one first piezo actuator of the longitudinal stroke device by (actuated) expansion and contraction thereof.

The at least one first piezo actuator is connected to the control unit. That is, the control unit is configured to actuate the at least one first piezo actuator in order to oscillate about a first piezo stroke or, respectively, to expand and contract. Thereby, the linear guide is arranged at the drive frame (essentially) parallel to the push rod. Thereby, the carriage is traversable along the linear guide for conveying in the longitudinal direction. The term conveying means especially a conveying drive of a medical-technical (syringe) pump by means of the piezoelectric drive unit according to the disclosure, whereby this may especially be (especially firmly) installable or, respectively, installed in the (syringe) pump.

Further, according to the disclosure, the control unit is thereby configured to coordinate (or, respectively, control) the first clamping device of the carriage. The first clamping device is configured (or, respectively, is controlled) to switch between a first coupling state for coupling of the carriage to the push rod and a first decoupling state for decoupling of the carriage from the push rod. The first coupling state is provided to generate during the first coupling state a carriage advance of the carriage according to the longitudinal stroke of the push rod movable from an axial starting position. The first decoupling state is provided to release the push rod for a motion separate from the carriage, especially for a retracting motion back to the starting position.

According to the disclosure, the first clamping device is thereby configured to have at least one second piezo actuator connected to the control unit for its actuating (or, respectively, operating, activation, piezoelectrically actuated oscillation). Thereby, the at least one second piezo actuator is configured to cause, by its expansion or contraction, at least the first coupling state. That is, the control unit is configured to actuate the at least one second piezo actuator in order to oscillate about a second piezo stroke or, respectively, to expand and contract.

The holding mechanism of the first clamping device functions by the (initially) advanced push rod being held (thereafter). The holding mechanism of the first clamping device is sufficient for the case that no further tensile forces act on the carriage, or, respectively, is especially sufficient to push the carriage back and forth without load. Thereby, the term of the carriage means the (bearing) main body that is axially movable or, respectively, traversable along the push rod.

The first clamping device is particularly distinguished from the prior art due to the advantage that the first clamping force to be applied or, respectively, applicable between the carriage and the push rod (in a cross direction to the longitudinal direction) can be effected directly, i.e. without (complex) force redirection.

Thereby, the term of the piezo actuator (or, respectively, piezo actuator) denotes a (piezoelectrics) component, which uses the (inverse) piezo effect to perform (in a defined manner) a mechanical motion or, respectively, deflection by applying of an electrical voltage. When the electrical voltage is applied to the piezo actuator (via a control unit), a motion amplitude or, respectively, a (relative) longitudinal expansion or, respectively, a so-called piezo stroke of the piezo actuator in the range of micrometers occurs as the displacement; whereby it contracts in cross direction. The displacement remains as long as the electrical voltage remains applied. Due to this, the motion amplitude or, respectively, (relative) longitudinal expansion of the piezo actuator relates to a reversible or, respectively, oscillating displacement.

In use of the piezo actuator, the motion amplitude or, respectively, the relative longitudinal extension of the piezo actuator is preferably controllable, especially controllable by applying of the electric voltage. In this respect, the motion amplitude or, respectively, the (relative) longitudinal extension is proportional to the electric field strength (where the field strength is defined as voltage/electrode distance quotient). Especially, the motion amplitude or, respectively, the relative longitudinal extension, i.e. an actual quantity or, respectively, an actual piezo stroke thereof, is controllable as a function of the electric voltage. That is, the piezo actuator is configured to map or, respectively, perform the motion amplitude or, respectively, the relative longitudinal extension as a complete or partial portion of a nominal or, respectively, theoretical or, respectively, maximum value of the (relative) displacement or, respectively, longitudinal extension of the piezo actuator.

Preferably, the piezo actuator may be designed as a multilayer piezo element (or, respectively, multilayer-element, piezoelectric stack, engl. “piezo stack”). For this purpose, several thin, especially ceramic, piezo elements with interposed electrodes are joined together to a series arrangement or, respectively, in sandwich construction.

Thereby, the control unit for controlling the piezoelectric drive unit may be formed or equippable with a memory unit or, respectively, a data medium (or, respectively, machine-readable storage medium). Preferably, the memory unit or, respectively, the data medium may be fixedly integratable or integrated. Alternatively or cumulatively, the control unit may be electrically (or, respectively, signal-technically) connected or connectable via an interface to a storage unit external with respect to the control unit.

According to the present disclosure, there are several advantages or, respectively, improvements compared to the prior art. Thus, the piezoelectric drive unit according to the disclosure is characterized by a high accuracy with, at the same time, high forces for conveying or, respectively, for precise, safe advancing and guidance in the drive. In this respect, a particularly simple and fast, yet at the same time sensitive recirculation measurement is proposed. Due to the piezo technology used throughout according to the disclosure, the number of moving components is advantageously reduced compared to the state of the art, which is accompanied by a reduced wear or, respectively, an increased robustness. In addition, this results, in a most favorable manner, in a reduced installation space of the drive unit. At the same time, it is possible to achieve these considerably advantageous optimizations with at least maintained or even higher performance as well as accuracy of the proposed drive unit.

As yet another advantage over conventional solutions ought it to be mentioned that the piezoelectric drive unit according to the disclosure enables a bi-directional drive mode. In this respect, a positive as well as negative carriage advance in the longitudinal direction is realizable or, respectively, traversable.

In addition, the piezoelectric drive unit according to the disclosure operates very quietly. This represents an important benefit for a user, especially for situations with infusions lasting several hours and/or with mobile carried (syringe) pumps. Furthermore, compared to conventional designs, the overall piezoelectric drive implementation offers the further product-related, not least with regard to mobile carried (syringe) pumps, very valuable benefits of a low installation space requirement as well as of a low energy consumption and of a low operational heat dissipation.

Preferably, alternatively or cumulatively, the at least one second piezo actuator may be configured to release, by its expansion, the first clamping device into the first decoupling state. Especially, the at least one second piezo actuator may be configured to cause the first decoupling state and/or to cause, by its contraction, the first coupling state.

Preferably, alternatively or cumulatively, the at least one second piezo actuator may be configured to act, by its expansion, by means of the first clamping device in a cross direction in a releasing and/or clamping manner on the push rod. Especially, the at least one second piezo actuator may be configured to provide a compressive force acting in the first clamping device or a resulting restoring force having a vector component acting in the cross direction on the push rod.

Preferably, alternatively or cumulatively, the control unit may be configured to provide the first coupling state and subsequently the first decoupling state. Thereby, the first coupling state is to be provided (or, respectively, is provided) in order to generate a carriage advance of the carriage according to the longitudinal stroke of the push rod movable from an axial starting position, especially to generate a carriage advance of the carriage to an advance position of the carriage (incremental, i.e. successively advanced) in longitudinal direction. Subsequently, the first decoupling state is to be provide (or, respectively, is provided) in order to release the push rod for a motion separate from the carriage. Especially, the separate motion may thereby relate to a retracting motion of the push rod back to its starting position while remaining of the carriage at the (incremental) advance position.

Preferably, alternatively or cumulatively, the piezoelectric drive unit may further comprise a guide rail and a second clamping device of the carriage. Thereby, the guide rail is provided parallel to the push rod at the drive frame. Thereby, the second clamping device of the carriage is actuatable by means of the at least one second piezo actuator and/or of at least one third piezo actuator connected to the control unit. Furthermore, the second clamping device is thereby configured to switch between a second coupling state and a second decoupling state. Thereby, the second coupling state is to be provided (or, respectively, is provided) to cause a run-inhibiting securing of the carriage at the guide rail against not intended (or, respectively, unintended) axial movements. Thereby, the second decoupling state is to be provided (or, respectively, is provided), to cause a run-free de-securing of the carriage from the guide rail. This serves in a particularly advantageous way for securing of the carriage against a slipping in the traversable longitudinal direction.

Particularly preferably, the guide rail may thereby be formed, in a radial direction (or, respectively, along or, respectively, at an acute angle to a cross direction), with a so-called line grind. This serves to build up an additional surface resistance for further securing against slipping of the clamped surfaces relative to each other.

The second clamping device, preferably provided in addition to the first clamping device, acts as a reinforcement of the transverse clamping of the carriage in order to secure it against further acting loads or, respectively, forces. The clamping takes place on the rib-shaped guide rail as a profile, preferably aluminum profile, provided for this purpose in the drive frame.

Thus, an advantageous benefit of the second clamping device is an additional securing of the carriage against axial slipping. When the carriage releases the push rod in the first decoupling state so that it can be retracted in order to then perform the next longitudinal stroke for the purpose of the incremental carriage advance (repeated cycle), the second clamping device ensures that when forces act on the carriage, it is still secured against slipping. This is advantageous, for example, in order to secure the presently disclosed piezoelectric drive unit or, respectively, the overall system of the drive unit installed in a (syringe) pump against off-plan mechanical loads. For example, the additional securing can prove to be of particular advantage for the scenario of an unintentional drop down or, respectively, of a fall of a patient with a mobile overall system.

Preferably, alternatively or cumulatively, the at least one first piezo actuator or, respectively, the (optional) at least one second or, respectively, the (optional) at least one third piezo actuator may be formed or, respectively, designed with different technical specifications (or, respectively, size, type, etc.). Especially, these possibly different technical specifications relate to constructionally relevant parameters such as a piezoelectrically actuated or, respectively, nominal piezo force and/or a piezoelectrically actuated or, respectively, nominal piezo stroke and/or dimensions (length, height, width) and/or material, coating, number of layers and the like. This serves a particularly compact construction and optimal, variable design of the function.

Preferably, alternatively or cumulatively, the (first/second/third) piezo stroke or, respectively, the (especially, but not limiting: unipolar) displacement or, respectively, the longitudinal extension, especially the nominal or, respectively, theoretical or, respectively, maximum value thereof, may be ca. 5 to 45 micrometers, further preferably ca. 8 to 16 micrometers, especially ca. 9 micrometers. Alternatively or cumulatively further preferably, the deflection (related to the length of the piezo actuator) or, respectively, the relative longitudinal expansion of the (first/second/third) piezo actuator, especially the nominal or, respectively, theoretical or, respectively, maximum value thereof, may be ca. 0.1%, especially ca. 0.2%.

Especially, the (first/second/third) piezo actuator may have a cross-sectional area of 5×5 mm²; a length of 9 mm; an electrical capacitance of 800 nF; and/or a piezo force of 850 Newton [e.g. piezo actuator of the technical type “PB 5.9” (Piezotechnics GmbH, Germany)]. Alternatively or cumulatively preferably, the piezo actuator may be at least partially metal-cased and/or formed with a protective coating, preferably polymeric or ceramic, which provides a protection against a high humidity.

Especially, the at least one (first/second/third) piezo actuator may mean a respective plurality or, respectively, a multiple of the (first/second/third) piezo actuator. Thereby, for example, a paired arrangement may be preferable, i.e. two (identically designed) first/second/third piezo actuators oscillate next to each other. In a corresponding manner, the piezo forces of the first/second/third piezo actuator are advantageously increased or, respectively, amplified by the multiple.

Especially, the second clamping device may be configured to provide the second coupling state without current. In other words, the second clamping device or, respectively, the carriage by means of this is secured in a run-inhibiting manner to the guide rail against unintended axial movements. This advantageously secures the piezoelectric drive unit against mechanical damage and/or unintended actuation (or, respectively, operation) by slipping. This serves especially to avoid in advance an unintentional, possibly prohibitive, drug administration to a patient, e.g. due to an unintentional drop of the (syringe) pump, which especially in the case of a mobile (syringe) pump carried for a long-term infusion relates to an accident risk to be mitigated.

Preferably, alternatively or cumulatively, the control unit may be configured to provide the second decoupling state respectively temporally precedingly or simultaneously to the first coupling state in order to perform a carriage advance of the carriage according to the longitudinal stroke of the push rod movable from an axial starting position.

Preferably, alternatively or cumulatively, the control unit may be configured to provide the second coupling state respectively temporally directly subsequently or simultaneously to the performed carriage advance.

Preferably, alternatively or cumulatively, at least one of the longitudinal stroke device and/or of the first clamping device and/or of the second clamping device may be formed as a respective solid body joint-supported mechanism. Especially, the respective solid body joint-supported mechanism may thereby be formed durable with respect to the oscillation of at least one (corresponding) piezo actuator from the at least one first, second and/or third piezo actuator. Thereby, the term of the solid body joint-supported mechanism means the use of solid body joints, i.e. elastically deformable or, respectively, deformed special areas of a (component) part for motion steering or, respectively, in the sense of functional kinematics. This feature serves the further increase of the accuracy and the robustness of the drive unit as a system or, respectively, an arrangement. In this respect, the solid body joint-supported mechanism is characterized by its clearance-free characteristic or, respectively, construction. Compared to the state of the art described in the introduction with a clearance-affected design based especially on slides, balls, springs, etc., the adjustment of the tolerances is much easier with the presently further preferred clearance-free design with solid body joints. This makes the design or, respectively, construction more cost-effective. In addition, the preferred embodiment is characterized by a far more compact design space and fewer parts. Especially, the further preferred durable (or, respectively, permanently elastic) forming or, respectively, design of the respective solid body joint-supported mechanism serves the wear and maintenance freedom or, respectively, the durability without phenomena of material fatigue. A mechanical load, a choice of material, a specific geometry as well as an experienceable (or, respectively, simulatable) deformation of the respective solid body joint can be incorporated into a corresponding design within the permissible stresses in the durable range (according to Wöhler curves). By the elastic deformation of the solid body joint of the solid body joint-supported mechanism, a restoring force results, as with a leaf spring. This has the advantage that it is possible to dispense with other restoring biassing elements, for example a spring, and with the bearing elements required for these, for example a bushing.

Preferably, alternatively or cumulatively, in the case that the first clamping device is formed as a solid body joint-supported mechanism, the first clamping device may comprise a bending spring. Thereby, the bending spring is arranged in a push rod clamping section between the push rod and the carriage. Further, the bending spring is rotatably supported on the drive frame in a bending spring pivot point in order to form a bending spring lever arm extending toward the push rod clamping section. Preferably, the bending spring may thereby further be restoringly counter-supported on the carriage in an abutment point in extension of the bending spring lever arm. Further, the bending spring is thereby formed to deform with piezoelectrically actuated expansion of the second piezo actuator in the cross direction such that the push rod is released from the carriage into the first decoupling state. Further preferably, the bending spring may thereby be formed to cause, with ist deformation, a preload with a bending spring restoring force resulting in the cross direction (such) that, with contraction of the second piezo actuator, the carriage couples back to the push rod into the first coupling state. The holding mechanism of the first clamping device functions by the (initially) advanced push rod being held (then) while the solid body joint returns into its original position.

Preferably, alternatively or cumulatively, in the case that the second clamping device is formed as a solid body joint-supported mechanism, it may have a pivot arm-like clamping lever. Thereby, the clamping lever is rotatably supported about the longitudinal axis of the linear guide in order to cause the second coupling state with piezoelectrically actuated expansion of the at least one second piezo actuator and/or third piezo actuator by its deflection force. Further preferably, the clamping lever may thereby be formed to translate a deflection force of the at least one second piezo actuator and/or third piezo actuator acting on the clamping lever into a guide rail clamping force, thereto amplified according to the lever law, applied to the guide rail. Preferably, a clamping lever may thereby be provided around a clamping lever pivot point, which coincides with a center of the linear guide. Especially, when the at least one third piezo actuator expands in cross direction, this causes a torsion of the clamping lever around the pivot point with a corresponding lever length of the clamping lever. Thereby, the lever length of the clamping lever corresponds to the distance from the clamping lever pivot point to a pressure point (when expanding) of the at least one third piezo actuator onto the clamping lever.

Preferably, alternatively or cumulatively, in the case that the longitudinal stroke device is formed as a solid body joint-supported mechanism, the longitudinal stroke device may comprise a double-sided lever rocker element. Thereby, the lever rocker element is, in its central lever rocker midsection at a rear end of the drive frame with respect to a direction of the carriage advance, especially at a rear frame plate, pivotally supported about a lever rocker rotation axis orthogonal to the longitudinal direction or, respectively, at a lever rocker pivot point. Thereby, the fixed lever rocker pivot point may preferably be provided in form of a screw connection of the solid body joint to the drive frame of the drive unit. The solid body joint, especially here the lever rocker element, is an element made of an elastic material, especially metal, which is always only elastically deflected or, respectively, deformed. Furthermore, the lever rocker element thereby forms for this purpose on both sides at the opposite ends a short lever rocker arm and a long lever rocker arm, respectively, in order to cause the longitudinal stroke of the push rod with piezoelectrically actuated expansion of the at least one first piezo actuator by a first piezo stroke.

Further preferably, the lever rocker element may thereby be formed to translate the first piezo stroke of the first piezo actuator applied to the short lever rocker arm into the longitudinal stroke of the push rod arranged on the long lever rocker arm, which is enlarged thereto according to the lever law.

A short lever rocker arm is formed as a distance from the lever rocker pivot point to a first piezo actuator. A long lever rocker arm is formed as a distance from the lever rocker pivot point to the push rod. Around the lever rocker pivot point, a lever with a short length of lever or, respectively, the short lever rocker arm is generated in a direction parallel to the longitudinal direction of the push rod by the first piezo stroke (e.g. ca. 0.01 mm) around the oscillating first piezo actuator. The short length of lever translates around the lever rocker pivot point with the long length of lever or, respectively, the long lever rocker arm to the push rod. Preferably, the short length of lever may be 4 mm to 10 mm, especially be ca. 7 mm. Preferably, the long length of lever may be 10 mm to 25 mm, further preferably 11 mm to 15 mm, especially ca. 12 mm. A lever effect of the solid body joint is effected according to a transmission ratio defined as quotient of the long length of lever to the short length of lever. Preferably, the transmission ratio as said quotient may lie in a range from 1.4 to 4, further preferably from 1.5 to 2.0, especially from 1.7 to 1.8.

According to said transmission ratio, a corresponding reduction of a force caused by the first piezo actuator results with simultaneous corresponding increase of the longitudinal stroke of the push rod acting in the longitudinal direction. The push rod stroke performs an incremental carriage advance in the longitudinal direction of the push rod. In other words, by the design of the transmission ratio or, respectively, of the short and/or of the long length of the lever, a predetermined longitudinal stroke of the push rod and/or a predetermined first piezo force applied by the first piezo actuator may be realized. Preferably, the first piezo force which acts axially on the push rod as conveying force for advancing may be at least 50 Newton, further preferably at least 100 Newton, especially at least 200 Newton.

A further-mentioned (or, respectively, second-mentioned) aspect of the present disclosure relates to a method for dispensing and/or dosing of medical liquids from a medical container using a piezoelectric drive unit according to the above first-mentioned aspect of the present disclosure. For avoidance of repetitions, reference is made to the foregoing disclosure relating to the first-mentioned aspect with respect to essential and preferred technical features of all further-mentioned aspects of the present disclosure. Thereby, the method according to the disclosure is executable via the control unit of the piezoelectric drive unit and thereby comprises subsequent steps, especially in manner of a repeatable step sequence:

An initial step relates to a coupling (or, respectively, a linkage, a clamping, temporarily connecting) of the carriage to the push rod by means of the first clamping device of the carriage into a piezoelectrically actuated first coupling state.

Thereafter, a further step relates to a generating of a carriage advance of the carriage, especially to an advance position incremental in longitudinal direction, coupled with a longitudinal stroke of the push rod from an axial starting position with acting of the longitudinal stroke device piezoelectrically actuated by means of the at least one first piezo actuator. Especially, the carriage advance may thereby relate to an advance position incremental in longitudinal direction.

Thereafter, a further step relates to a decoupling (or, respectively, an uncoupling, release, temporary release of the clamping) of the carriage from the push rod by means of the first clamping device of the carriage into a piezoelectrically actuated first decoupling state.

Thereafter, a further step relates to a separate motion of the push rod with acting of the longitudinal stroke device piezoelectrically actuated by means of the at least one first piezo actuator. Especially, the separate motion may relate to a retracting motion of the push rod from the advance position incremental in the longitudinal direction back to the starting position.

Preferably, alternatively or cumulatively, especially in the case of the piezoelectric drive unit preferably provided with the second clamping device of the carriage, the method (further advantageously developed beyond the basic principle) may comprise at least one of the two further optional steps executable via the control unit, or both, as follows:

An initial step relates to a coupling (or, respectively, a linkage, a clamping, temporarily connecting) of the carriage to the push rod by means of the first clamping device of the carriage into a piezoelectrically actuated first coupling state.

Thereafter, an optional step, executable by means of the, if applicable, provided second clamping device of the carriage, relates to a switching from a second coupling state for run-inhibiting securing of the carriage at the, if applicable, provided guide rail into a piezoelectrically actuated second decoupling state for run-free de-securing of the carriage from the guide rail.

Thereafter, a further step relates to a generating of a carriage advance of the carriage, especially to an advance position incremental in longitudinal direction, coupled with a longitudinal stroke of the push rod from an axial starting position with acting of the longitudinal stroke device piezoelectrically actuated by means of the at least one first piezo actuator. Especially, the carriage advance may thereby relate to an advance position incremental in longitudinal direction.

Thereafter, an optional step, executable by means of the, if applicable, provided second clamping device of the carriage, relates to a switching from the second decoupling state back into the piezoelectrically actuated second coupling state for run-inhibiting securing of the carriage to the, if applicable, provided guide rail, especially on the incremental advance position. Regarding last-mentioned step, it should be noted that by the term of the (back-)switching it is meant that in the case of the, if applicable, provided second clamping device of the carriage reasonably both corresponding optional steps are performed (or both are not performed, insofar as the second clamping device e.g. temporarily could not be used). That is, that the one first-mentioned optional step of the switching into the second decoupling state (starting from a state in the second coupling state) is accompanied by or, respectively, is functionally related to the second-mentioned optional step of the (back-)switching into the piezoelectrically actuated second coupling state, especially if related to the repeatable step sequence.

Thereafter, a further step relates to a decoupling (or, respectively, an uncoupling, release, temporary release of the clamping) of the carriage from the push rod by means of the first clamping device of the carriage into a piezoelectrically actuated first decoupling state.

Thereafter, a further step relates to a separate motion of the push rod with acting of the longitudinal stroke device piezoelectrically actuated by means of the at least one first piezo actuator. Especially, the separate motion may relate to a retracting motion of the push rod from the advance position incremental in the longitudinal direction back to the starting position.

Preferably, alternatively or cumulatively, the method may comprise a further step executable via the control unit as follows: A controlling of the oscillation of at least one piezo actuator from the at least one, second and/or third piezo actuator. Thereby, especially a (step of the) controlling of the longitudinal stroke of the push rod movable in an oscillating manner at drive frame by means of the longitudinal stroke device may be performed. Further preferably, controlling may be steplessly formed.

A further-mentioned aspect of the present disclosure relates to a medical-technical (syringe) pump having a piezoelectric drive unit according to the first-mentioned aspect of the present disclosure. Alternatively or cumulatively, the medical-technical pump may be formed and configured to execute the method for dispensing and/or dosing of medical liquids according to the second-mentioned aspect of the present disclosure. Especially, the medical-technical (syringe) pump may thereby be configured as a mobile infusion pump portable by the user.

A further-mentioned aspect of the present disclosure relates to a first clamping device and/or the second clamping device for a piezoelectric drive unit according to the first-mentioned aspect of the present disclosure. Especially, the first clamping device and/or the second clamping device may thereby be formed for use as corresponding component (accessory part or, respectively, retrofit part or, respectively, spare part). Alternatively or cumulatively, the first clamping device and/or the second clamping device may thereby be formed for use in the method for dispensing and/or dosing of medical liquids according to the above second-mentioned aspect of the present disclosure.

A further-mentioned aspect of the present disclosure relates to a computer program on a data medium (or, respectively, machine-readable storage medium), configured for performing the method for dispensing and/or dosing of medical liquids according to the disclosure, whereby the method comprises or, respectively, executes the steps executable via the control unit, especially the repeatable step sequence.

In summary, the use of a drive unit realized entirely by means of piezo actuators (as such powerful, permanently precise, wear-free), i.e. especially as replacement or, respectively, by omitting of the motor-driven spindle, stride and/or oscillating drive usually used in the drive unit, in a syringe pump represents an essential idea of the present disclosure. An (piezoelectrically) axially oscillating (or, respectively, oscillating in longitudinal direction) push rod (whose longitudinal stroke excited by the at least one first piezo actuator) pushes or pulls a carriage in the desired (advance) direction. For this purpose, the push rod (or, respectively, the longitudinal stroke device) as well as the first clamping device or, respectively, preferably further the second clamping device are each moved by means of the corresponding at least one (first/second/third) piezo actuator.

Preferably, alternatively or cumulatively, this actuating (or, respectively, operating, activation, piezoelectrically actuated oscillation), depending on the amplitude level of the oscillation, may be performed in steplessly adjustable accuracy (up to the nominal maximum piezo stroke of the corresponding, respectively used (first/second/third) piezo actuator. Consequently, it is of particular advantage that especially the accuracy of the actuator (the smallest possible longitudinal stroke of the push rod or, respectively, carriage advance) may be steplessly adjustable via the (piezoelectrically actuated) deformation of the first piezo actuator.

In a particularly preferred embodiment, the different (the at least one first or, respectively, second or, respectively, third) piezo actuator(s) move, so to speak in a functional or, respectively, mechanical interaction, by means of different (of the first and the second) clamping devices for alternately securing of the carriage on the one hand to the axially-oscillated push rod and on the other hand to the guide rail as a profile fixed via the drive frame. With suitable switching via the control unit, the carriage is then moved or, respectively, (stepwise, clockwise, cyclewise) traversed by the desired carriage advance in longitudinal direction. In other words, by ‘re-gripping’ effected by means of the control unit in manner of a coordinated temporary (or, respectively, phased) clamping or, respectively, coupling of the carriage at the push rod and/or at the guide rail, an (incremental) carriage advance in the desired longitudinal direction is generated or, respectively, a (drive) motion results.

Preferably, alternatively or cumulatively, the longitudinal stroke device) and/or the first clamping device and/or the second clamping device may be formed as the solid body joint-supported (lever) mechanism(s). This allows in a particularly advantageous way to convert the in the micrometer range small piezo strokes of the respective corresponding (first/second/third) piezo actuator (even in continuous operation) in a clearance-free manner into larger (useful) strokes. Further is to be seen as particularly advantageous that by means of the preferred solid body joint-supported (lever) mechanism(s), sufficient holding forces are generated with regard to the traversable components or, respectively, the carriage, in accordance with the very high requirements for the operational safety of such medical-technical drive units.

In addition, the drive unit proposed according to the disclosure may be easily designed as passively safe, i.e. de-energized locked, in order to effectively secure a medical-technical (syringe) pump in advance against an unintended drug delivery.

In general, as already explained above, this results in manyfold technical advantages which can be attributed to the solid-state physics-based, permanent ultra-fine precision of the piezoelectrically induced oscillation or, respectively, expansion, even at most minute motion amplitudes or, respectively, piezo strokes of the (first/second/third) piezo actuator. The small size of the piezo actuator also helps to reduce the overall size of the drive unit and accordingly of the thus resulting (syringe) pump. With regard to a syringe pump, a slow advance or, respectively, traveling of the piston plunger (few millimeters per hour) may be preferred. Thereby, in a stable, clean, quiet operation, for example, a conveying rate of minimum 0.01 ml per hour may be provided. Due to the already explained energy efficiency according to the disclosure, a longer up to long carrying time, e.g. spanning several days, in the case of a mobile (i.e. carried along/worn by a patient) pump is feasible to his convenience.

The person skilled in the art thereby understands that on the basis of the present disclosure other fields of application than for a medical-technical infusion pump are conceivable. While a focus of the conceivable areas of application of the present disclosure lies in the precise dosing of an infusion solution, supplied in a prefabricated manner in the application form of a syringe, of an already diluted at least one active ingredient or, respectively, drug over a longer period of time, further possible applications or, respectively, uses of the present disclosure are obvious to the person skilled in the art. Thus, this relates to manifold objects of the automatic fine dosing of flowable liquids in the production, for example in the perfume blending, manufacturing of pharmaceuticals, etc. Especially, it is conceivable, with appropriate adjustment of the dimensioning or, respectively, of the technical parameters of the presently disclosed drive unit, to carry out a precise dosing of a (more) concentrated active substance or, respectively, drug into a solution, especially into a stabilizing formulation and/or into an infusion solution, in a diluting manner. For example, this may be useful in a surgical situation with regard to an anesthetic (measure) to be basically or, respectively, acutely performed and/or to a life-sustaining stabilization of a patient. Especially, the drive unit may also be optimized to this effect to perform a punctual and/or spontaneous fine dosage in specifically desired or, respectively, piezoelectrically controllable dosage volumes. Furthermore, it is conceivable to use in a medical-technical manner the precise drive unit, especially with reversal of the above-mentioned advance direction in longitudinal direction of the push rod, for specific automatic sampling or, respectively, blood collection. Especially, modifications for the use as precise, wear-free drive unit of smallest size for dosing pumps or, respectively, application pumps for (automated) (liquid) mixing, analytics, manufacturing, assembly, are thus obvious.

### BRIEF DESCRIPTION OF THE DRAWINGS

The disclosure is described in more detail below based on preferred embodiments with reference to the accompanying drawings.

FIG. 1 is a perspective view of a piezoelectric drive unit for a medical-technical syringe pump (itself not shown) according to a first preferred embodiment according to the present disclosure;

FIG. 2 is a further perspective view (rotated in space to the view of FIG. 1) of the piezoelectric drive unit according to the first embodiment;

FIG. 3a or, respectively, 3b are two respective plan views of the piezoelectric drive unit according to the first embodiment, illustrating a piston plunger travel path of the drive unit, relevant in use in the corresponding medical-technical syringe pump;

FIGS. 4b to 4d (with reference to the view section line B-B indicated in the plan view of FIG. 4a) are different illustrations of a longitudinal stroke device of the piezoelectric drive unit according to the first embodiment, as exemplarily formed here as a preferred solid body joint-supported mechanism:

FIG. 4b is a cross-section view in sectional plane B-B;

FIG. 4c is a corresponding rear view; and

FIG. 4d is a perspective plan view on another sectional plane for illustration of the kinematic operation of the longitudinal stroke device, piezoelectrically actuated by means of a first piezo actuator;

FIG. 5 is an enlarged detail of the perspective view of FIG. 2 (rotated in space for this purpose) of the piezoelectric drive unit according to the first embodiment, focusing on the region of the longitudinal stroke device for a push rod;

FIG. 6 is an enlarged detail of the perspective view of FIG. 1 (rotated in space for this purpose) of the piezoelectric drive unit according to the first embodiment, focusing on the region of a second clamping device for a carriage traversable on the push rod, configured for its run-inhibiting securing to a guide rail;

FIG. 7 is a perspective view of a drive frame of the piezoelectric drive unit according to the first embodiment (shown isolated in manner of an exploded view and in a profile-like cross-section), especially showing the guide rail;

FIGS. 8a to 8c (with reference to the view section line A-A indicated in the plan view of FIG. 4a) are different illustrations of a first clamping device as well as of a second clamping device of the carriage of the piezoelectric drive unit according to the first embodiment, as exemplarily formed here as further two preferred solid body joint-supported mechanisms:

FIG. 8a is a cross-section view in sectional plane A-A (see FIG. 4a), showing the first clamping device as well as the second clamping device;

FIG. 8b is a corresponding detail section C from FIG. 8a regarding the second clamping device; and

FIG. 8c is a corresponding detail section D from FIG. 8a regarding the third clamping device;

FIG. 8d is another (essentially corresponding to FIG. 8a) cross-section view for illustration of the kinematic operations of the first clamping device, piezoelectrically actuated by means of a second piezo actuator, or, respectively, of the second clamping device, piezoelectrically actuated by means of a third piezo actuator, of the piezoelectric drive unit according to the first embodiment;

FIG. 9 is a second embodiment of the piezoelectric drive unit according to the disclosure, for schematic illustration of the temporal sequence or, respectively, coordination of (working) steps executable by means of a control unit in the basic principle, i.e. when considering only one first clamping device; or, respectively, for schematic illustration of a corresponding method (with respect to essential steps of a cycle) for dispensing and/or dosing of medical liquids; and

FIG. 10 is a third embodiment of the piezoelectric drive unit according to the disclosure, for schematic illustration of the temporal sequence or, respectively, coordination of (working) steps executable by means of a control unit, illustrating the case of a drive unit identical or comparable to the first embodiment (cf. FIGS. 1 to 8d) with a preferred second clamping device; or, respectively, for schematic illustration of the corresponding method (including optional, i.e. preferably executable, steps of a cycle); whereby otherwise comparable conditions exist in relation to the second embodiment in FIG. 9.

The Figures are of a schematic nature only and serve solely for the purpose of the understanding the disclosure. The same elements are designated with the same reference signs.

### DETAILED DESCRIPTION

The following embodiments of the present disclosure are described on the basis of the corresponding figures.

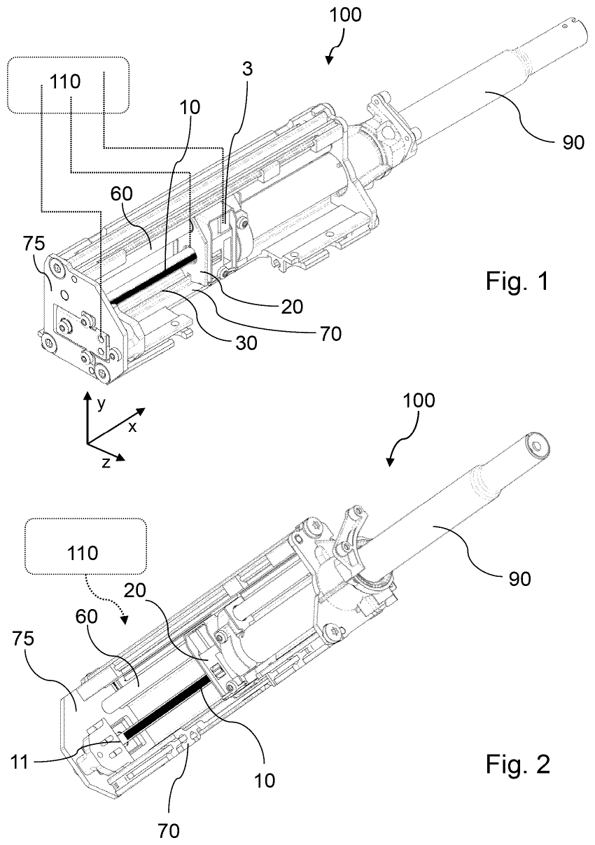

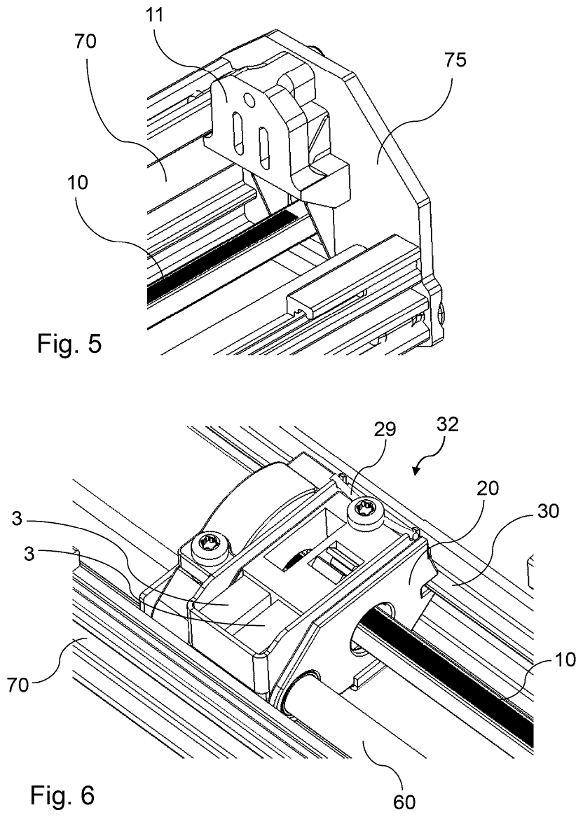

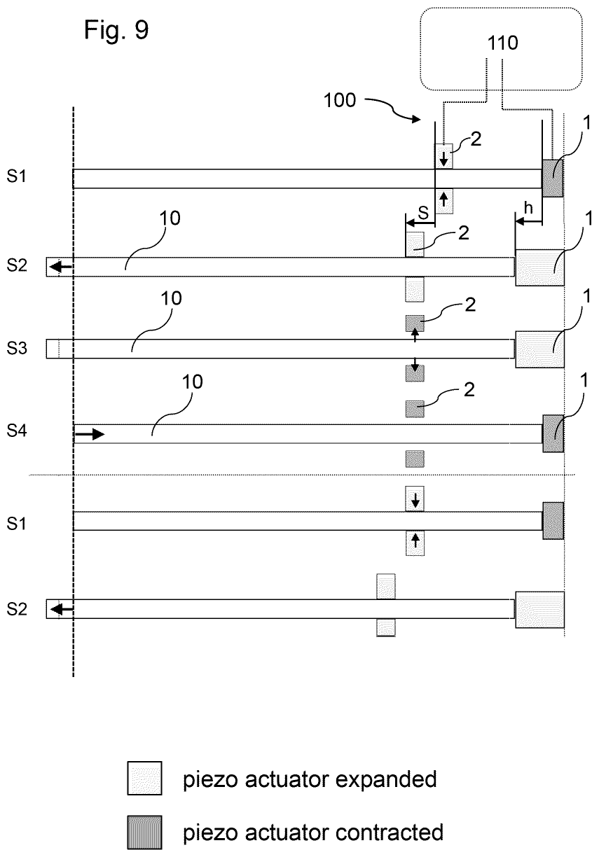

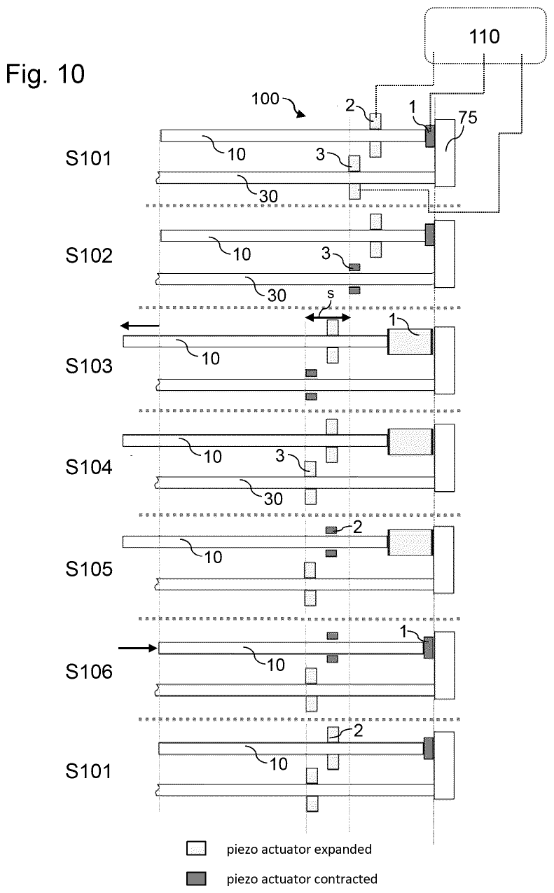

FIGS. 1 to 8d relate to a piezoelectric drive unit 100 for a medical-technical syringe pump (not shown; for example, an infusion pump marketed under the brand name “Space Plus Perfusor” of the present applicant B. Braun) according to a first preferred embodiment according to the present disclosure. Then again, FIGS. 9 and 10 schematically illustrate (in the basic principle, i.e. with the patent legal character of a general disclosure) the temporal sequence or, respectively, coordination of (working) steps executable by means of a control unit 110. Especially, the extended scheme of FIG. 10 illustrates a method corresponding to the first preferred embodiment.

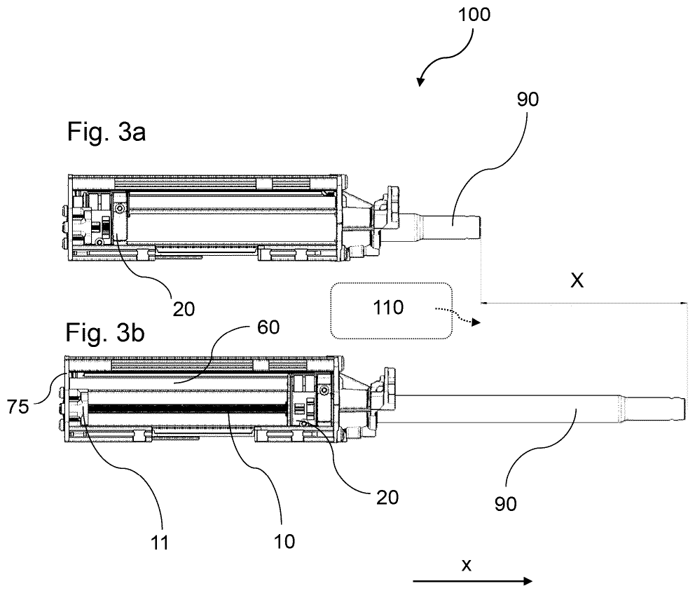

As can be seen from the overall views of a piezoelectric drive unit 100 in the FIGS. 1 to 4d, this is formed with a piston plunger 90 for the axial (longitudinal) drive of a (not illustrated) medical-technical syringe pump or comparable pump for dispensing and/or dosing of medical liquids. Thereby, the piston plunger 90 is traversable in the (axial) longitudinal direction x about a (total or, respectively, cumulative) piston plunger travel path X (see especially FIGS. 3a and 3b). For this purpose, the piston plunger 90 is moved stepwise (or, respectively, clockwise, cyclewise) in a plurality of incremental advances (cf. with reference to the FIGS. 9 and 10: of carriage advances s).

The piezoelectric drive unit 100 (FIGS. 1 to 8d) further has a drive frame 70 with a (rear) frame plate 75 at an end opposite the piston plunger 90. The drive frame 70 functions in the sense of a fixed profile or of a stable outer base housing. The piezoelectric drive unit 100 further comprises a linear guide 60. Thereby, the linear guide 60, designed in the form of a guide shaft, is chucked in the drive frame 70 in the longitudinal direction x.

Thus, along the linear guide 60, a carriage 20 movably arranged (on it) can be traversed in order to realize the actual advance function for conveying. Together with the carriage 20, a first clamping device 21 of the carriage is also traversed.

The piezoelectric drive unit 100 further comprises a push rod 10. The push rod 10 is arranged at the end at the drive frame 70 or, respectively, at the frame plate 75 via a longitudinal stroke device 11. Thereby, the linear guide 60 and the push rod 10 are parallel to each other (essentially, within the usual positional tolerances).

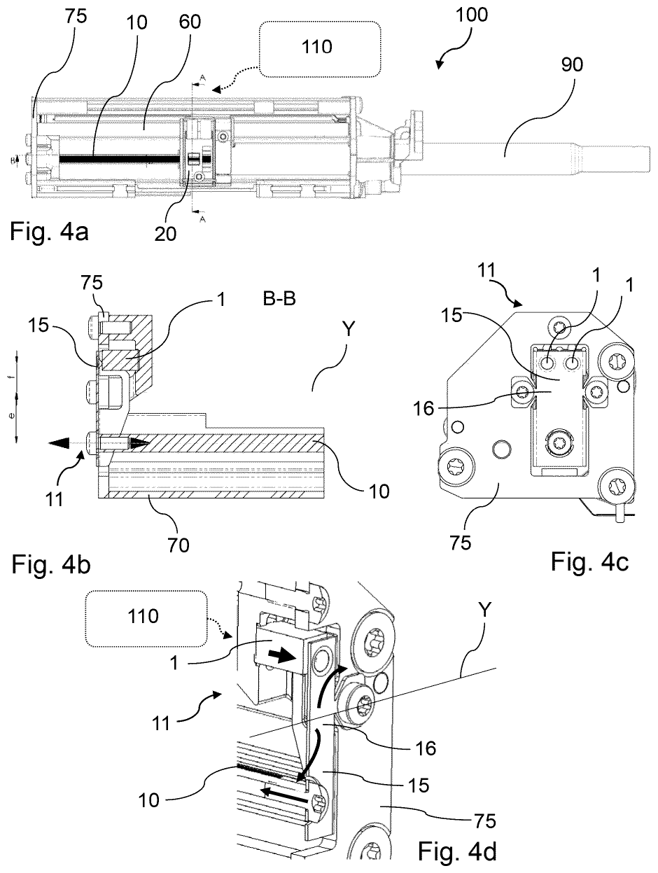

The piezoelectric drive unit 100 further comprises (at least) a first piezo actuator 1 of the longitudinal stroke device 11, a second piezo actuator 2 of the first clamping device 21 and a third piezo actuator 3 of a second (optional) clamping device 32. That is, the first piezo actuator 1 (esp. FIG. 4c), the second piezo actuator 2 and the third piezo actuator 3 (esp. FIG. 6) each consist of two such ones side by side, i.e. in the manner of a first/second/third double piezo actuator.

The respective first/second/third piezo actuators 1, 2, 3 are connected to a control unit 110 (indicated or, respectively, drawn in as a dashed outlined symbol box) for piezoelectrically actuated expansion and contraction by a first/second/third piezo stroke. For this purpose, the controlling from the control unit 110 takes place in the respective Figures via the control connections shown as dashed lines or, respectively, arrows (cf. FIGS. 1, 1, 3a/b, 4a/d, 8a/d; 9 and 10). Thereby, the first/second/third piezo stroke is preferably steplessly adjustable (in dependence of a respective control voltage applied).

The push rod 10 is configured by means of the longitudinal stroke device 11 to be movable in an oscillating manner by a longitudinal stroke h in the longitudinal direction x or, respectively, is (longitudinally) oscillated or, respectively, moved back and forth. Thereby, at least one first piezo actuator 1 of the longitudinal stroke device 11 acts on the push rod 10. Thus, the carriage 20 is moved along the linear guide 60 due to the first piezo force converted by means of the longitudinal stroke device 11 by the carriage 20 being pushed (or, respectively, pulled back) from the push rod 10.

As can be seen from FIGS. 4b to 4d (with reference to the view section line B-B indicated in the plan view of FIG. 4a), the longitudinal stroke device 11 is formed with a double-sided lever rocker element 15 (as a preferred solid body joint-supported mechanism). The lever rocker element 15 is, in its central lever rocker midsection 16 on the frame plate 75, pivotally supported about a lever rocker rotation axis Y orthogonal to the longitudinal direction x. At the opposite ends of the (double-sided) lever rocker element 15, a short lever rocker arm f and a long lever rocker arm e are formed, respectively.

With piezoelectrically actuated expansion of the at least one first piezo actuator 1 by a first piezo stroke, the longitudinal stroke h of the push rod 10 is to cause (cf. FIGS. 9 and 10). FIG. 4d ought thereby to illustrate the kinematic functionality or, respectively, the lever translation by means of arrows. For example, the maximum conveying force required for a syringe pump or, respectively, common infusion syringes may be ca. 100 Newton. The push rod 10 is designed to apply this conveying force. For example, piezo actuators selected for the (at least one) first piezo actuator are specified by the manufacturer at maximum 850 Newton piezo force of and a maximum piezo stroke of 0.009 mm. By means of the lever rocker element 15, the force (piezo force converted to push rod force) is now reduced and at the same time the stroke (piezo stroke converted to longitudinal stroke as push rod working stroke) increased. Here, according to the lever law, the two lengths of lever of the long lever rocker arm e and of the short lever rocker arm f engage. In the FIGS. 4a to 4d, an exemplary force lever reduction of 1:2 is illustrated.

The first clamping device 21 of the carriage 20 is controlled by the control unit 110 via the second piezo actuator 2. After completed clamping, the carriage 20 can be moved together with the (travelling) push rod 10 in a (temporary) first coupling state. Consequently (cf. FIGS. 9 and 10), a carriage advance of the carriage takes place according to the longitudinal stroke of the push rod. In order to enable the push rod 10 to perform a separate retracting motion, independent of the carriage 20, back to its starting position, without retracting the carriage 20 again or, respectively, undesirably reversing the desired carriage advance, the first clamping device 21 is set back into a first decoupling state.

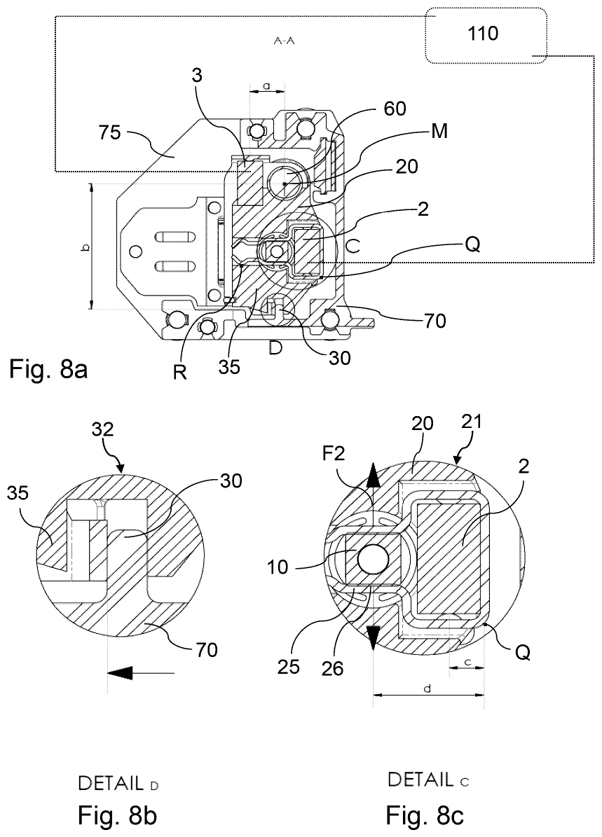

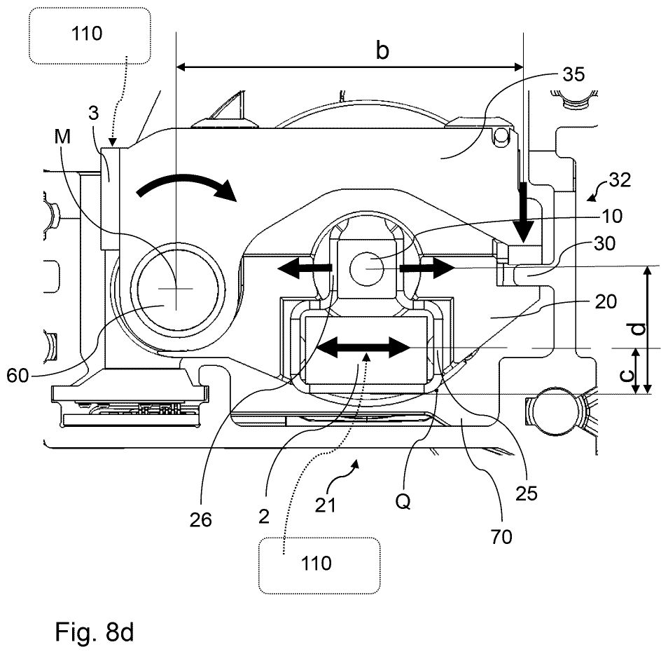

As can be seen especially from FIGS. 8a (with reference to the view section line A-A indicated in the plan view of FIG. 4a), 8c, 8d, the first clamping device 21 is formed with a bending spring 25 (as a preferred solid body joint-supported mechanism). The bending spring 25 is arranged in a push rod clamping section 26 between the push rod 10 and the carriage 20. Further, the bending spring 25 is thereby rotatably supported on the drive frame 70 in a bending spring pivot point Q in order to form a bending spring lever arm d extending toward the push rod clamping section 26. The bending spring 25 is further restoringly counter-supported at the carriage 20 in an abutment point R in extension of the bending spring lever arm d. With or, respectively, by expansion of the second piezo actuator 2 in the cross direction (see FIG. 1: y, z), the bending spring is deformed such that the push rod 10 is decoupled or, respectively, released from the carriage 20. Thereby, the deformation also causes a preload with a bending spring restoring force resulting in the cross direction (reaction to F2, as indicated by the arrows), so that with contraction of the second piezo actuator 2 the carriage 20 is coupled or, respectively, clamped back to the push rod 10 into the first coupling state.

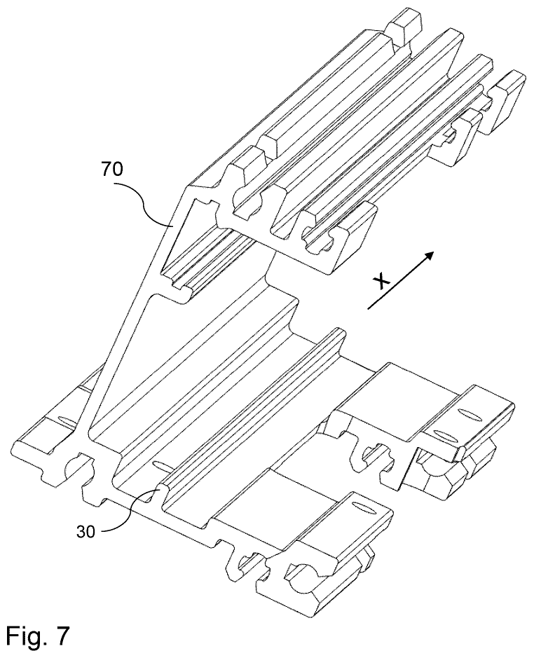

The piezoelectric drive unit 100 according to the preferred first embodiment further comprises a guide rail 30 and a second clamping device 32 of the carriage 20. The guide rail 30 is provided in the longitudinal direction x or, respectively, parallel to the push rod 10 at the drive frame 70 (especially FIG. 7). The second clamping device 32 of the carriage 20, as especially shown in FIGS. 6, 8a, 8b and 8d, is actuated by means of the third piezo (double) actuator 3 in order to switch between a second coupling state and a second decoupling state. For the temporary time period of the second coupling state (but especially also occurring without current), the carriage 20 is run-inhibitingly secured or, respectively, blocked at the guide rail 30 against unintended axial movements. For the temporary time period of the second decoupling state, the carriage 20 is released or, respectively, unblocked from the guide rail or, respectively, the previously existing blockade for free running in the longitudinal direction is removed again.

For a design of the second clamping device 32, preferably not the conveying force, but the possible acting force in the case of a drop may be assumed. In this respect, in such a scenario, breaking loose of the carriage 20 must not occur. For example, with the following assumptions of such a scenario: a drop height of ca. 1 m, a weight of a syringe pump of ca. 2 kg and an impact duration of 10 ms, using the force impact equation, a force acting during an impact can be estimated at ca. 886 Newton. At least this clamping force, especially greater than or equal to 1,000 Newton, must be applied by first and/or second clamping device(s) 21, 32 (alone or together).

Since, in addition, a minimum value for a (second and/or third) piezo stroke (due to elasticities in the respective clamping mechanism) must also be realized in order to ensure reliable opening and closing, two (second or, respectively, third) piezo actuators are used in each case and their piezo force is connected in parallel as (second or, respectively, third) double piezo actuator (not visible in FIGS. 8a, 8c, 8d due to the views in a cross section).

As can be seen from FIGS. 6, 8a (with reference to the view section line A-A indicated in the plan view of FIG. 4a), 8b, 8d, the second clamping device 32 is formed with a pivot arm-type clamping lever 35 (as a preferred solid body joint-supported mechanism). The clamping lever 35 is rotatably supported about the longitudinal axis M (especially FIG. 8d) of the linear guide 60. The piezoelectrically actuated expansion of the third piezo actuator 3 causes the second coupling state by its deflection force or, respectively, third piezo force acting on the clamping lever 35 (cf. the deflection in the direction of rotation indicated by means of the arrow at the top left in FIG. 8d). The third piezo force is converted into a guide rail clamping force, thereto amplified according to the lever law, applied to the guide rail 30.

As can be derived from FIG. 6, the carriage 20 is arranged in an outer sheet metal body. A carriage pressing element 29 is arranged as a bow-shaped wire spring (top in FIG. 6) secured by means of the Torx screws. The carriage pressing element 29 effects that the sheet metal body is always connected without clearance to the transversely oscillating third piezos 3 (expansion to the top right in FIG. 6). When the pair of third piezos 3 expands, the carriage pressing element 29 is pretensioned in order to exert downward counterforces. In this respect, the (bow-shaped) carriage pressing element 29, in its effort to align itself in a straight line, causes a restoring pressing force against the carriage 20 or, respectively, a counterforce to the third piezo force resulting from the expansion of the third piezos 3. In other words, the spring force of the (bow-shaped) carriage pressing element 29 acts downwards or, respectively, against the sheet metal body, by which the guide rail clamping force serving for securing of the carriage 20 is generated.

FIG. 9 or, respectively, FIG. 10 show a second or, respectively, third embodiment of the piezoelectric drive unit according to the disclosure as well as of the corresponding method. Herein, the temporal sequence or, respectively, coordination of (working method) steps executable by means of a control unit 110 (filled in or, respectively, illustrated with a dotted line) in a (repeatable) basic cycle S1 to S4 or, respectively, in an extended cycle S101 to S106 is schematically (or, respectively, in principle) illustrated. Thereby, the control unit 110 is configured to actuate (or, respectively, to operate, to activate) the at least one first/second/if applicable, third piezo actuator, respectively (via the control connections indicated by dashed-lined lines) (for piezoelectric oscillation).

In short, the basic cycle S1 to S4 relates to an alternating coordination in which, on the one hand, either the carriage moves or does not move and, on the other hand, the push rod 10 is clamped or not clamped by the carriage.

Insofar as the sequence shown in FIG. 10 relates to the cycle extended by the optional steps S102 and S104, FIG. 10 corresponds especially with the first embodiment (cf. FIGS. 1 to 8d) of the piezoelectric drive unit according to the disclosure, which additionally has the preferably provided second clamping device (securing to the guide rail 30). For reasons of a clear illustration, in FIGS. 9 and 10 the longitudinal stroke device 11, the first clamping device 21 and, if applicable, the second clamping device 32 (reference signs with reference to FIGS. 1 to 8d emphasizing the structural device aspects) are not shown as such, but only one of the corresponding first piezo actuator 1, one of the corresponding second piezo actuator 2 and, if applicable, one of the corresponding third piezo actuator 3 illustrated. In this respect, the (relative or, respectively, incremental) position (on the horizontally drawn travel axis) of the (likewise not shown) carriage (20) can be derived from the (relative or, respectively, incremental) position of the second piezo actuator 2 (if applicable, together with that of the third piezo actuator 3):

Initially (or, respectively, at each new cycle start of the repeatable cycle S1 to S4; S101 to S106), a step S1, S101 of coupling/clamping of the carriage to the push rod 30 takes place. Thereby, the carriage is clamped to the push rod 30 by means of piezoelectrically actuated expansion of the second piezo actuator 2 (see vertical arrows in FIG. 9). This corresponds to the (temporary) first coupling state of the first clamping device of the carriage.

In short, in step S1, S101 the push rod 10 is gripped by the carriage (clamped by means of second piezo actuator 2).

Thereafter, optionally (only FIG. 10) a step S102 of a switching from a second coupling state (run-inhibiting securing of the carriage to the guide rail 30) into a (temporary) second decoupling state (run-free de-securing of the carriage from the guide rail 30) takes place. For this purpose, the third piezo actuator 3 (the second clamping device of the carriage) is piezoelectrically actuated for expansion, so that the previously existing clamping is released in a run-free manner.

In short, in (optional) step S102, the clamping or, respectively, running restraint of the carriage on the guide rail 30 is removed or, respectively, released.

Thereafter, a further step S2, S103 (of a generating) of a carriage advance s of the carriage takes place, which is activated or, respectively, caused by a longitudinal stroke h of the push rod (see in FIG. 9/10 the horizontal arrow as well as the fine-dashed vertical line located on the left of the diagram). For this purpose, the first piezo actuator 1 of the longitudinal stroke device expands from an axial starting position. It should be noted that in the schematic illustration of FIGS. 9, 10, the first piezo stroke coincides with the longitudinal stroke h. This means that a preferably vertical line on the left-hand side of the figure is not possible. I.e. any preferably provided lever conversion (cf. FIGS. 4a to 4d) is neglected herein; analogous applies to the piezo actuators 2, 3.

In short, in step S2, S103 the push rod 10 performs longitudinal stroke h by being advanced by a first piezo stroke of the first piezo 1, and thereby takes the pre-clamped carriage with it.

Thereafter, optionally, a step S104 (FIG. 10 only) of (back-)switching from the second decoupling state (run-free de-securing of the carriage from the guide rail 30) into the (temporary) second coupling state (run-inhibiting securing of the carriage to the guide rail 30) takes place. For this purpose, the third piezo actuator 3 (of the second clamping device of the carriage) is piezoelectrically actuated for contraction. Due to the coupling/clamping of the carriage to the guide rail 30, the carriage is secured against slipping.

In short, in (optional) step S104, the carriage is clamped on the guide rail 30 or, respectively, secured or, respectively, inhibited from running.

In a subsequent step S3, S105, a decoupling of the carriage from the push rod 30 takes place. For this purpose, the carriage is released from the push rod 30 by means of piezoelectrically actuated contraction of the second piezo actuator 2 (see vertical arrows in FIG. 9). This corresponds to the first (temporary) decoupling state of the first clamping device of the carriage.

In short, in step S3, S105 the carriage releases its clamping to the push rod 30.

Subsequently, in a step S4, S106 of a motion of the push rod 10 separate from the carriage 10, the push rod 10 is moved or, respectively, pulled back to its starting position (see in FIG. 9/10 the horizontal arrow as well as the fine-dashed vertical line located on the left of the diagram). For this purpose, the first piezo actuator 1 of the longitudinal stroke device contracts back to the axial starting position.

In short, in step S4, S106 the push rod 10 is again retracted to its starting position, while the carriage remains at a new (incremental) advance position. Compare the offset of the carriage according to that of the second piezo actuator 2 at the beginning of the new cycle at the repeated step S1 or, respectively, S101 (bottom of diagram) compared to its position at the beginning of the first cycle (top of diagram).

By (multiple) repetition of the cycle, the incremental carriage advances s add up or, respectively, accumulate. Thus, in sum, the piston plunger travel path X results (with reference to FIGS. 3a/3b).

1.-15. (canceled)

16. A piezoelectric drive unit for dispensing and/or dosing of medical liquids from a medical container, the piezoelectric drive unit comprising:
a drive frame;
a control unit;
a push rod configured at the drive frame to be movable in an oscillating manner about a longitudinal stroke in a longitudinal direction by a longitudinal stroke device to be moved back and forth with acting of at least one first piezo actuator of the longitudinal stroke device connected to the control unit by expansion and contraction of thereof;
a linear guide arranged at the drive frame parallel to the push rod;
a carriage traversable along the linear guide for conveying in the longitudinal direction; and
a first clamping device of the carriage, coordinated by the control unit, which is configured to switch between a first coupling state for coupling of the carriage to the push rod and a first decoupling state for decoupling of the carriage from the push rod,
the first coupling state being provided to generate a carriage advance of the carriage, according to the longitudinal stroke of the push rod movable from an axial starting position, to an advance position of the carriage incremental in longitudinal direction,
the first decoupling state being provided to release the push rod for a motion separate from the carriage for its retracting motion back to the axial starting position while remaining of the carriage at the advance position, and
the first clamping device comprising at least one second piezo actuator connected, for its actuating, to the control unit, whereby the at least one second piezo actuator is configured to cause, by expansion or contraction thereof, at least the first coupling state.

17. The piezoelectric drive unit according to claim 16, wherein the at least one second piezo actuator is configured to release, by expansion of the second piezo actuator, the first clamping device into the first decoupling state and/or to cause, by contraction of the second piezo actuator, the first coupling state.

18. The piezoelectric drive unit according to claim 16, wherein the at least one second piezo actuator is configured to act, by expansion of the second piezo actuator, by the first clamping device in a cross direction in a releasing and/or clamping manner on the push rod.

19. The piezoelectric drive unit according to claim 16, wherein the piezoelectric drive unit further comprises:
a guide rail provided parallel to the push rod at the drive frame; and
a second clamping device of the carriage, which is actuatable by the at least one second piezo actuator and/or of at least one third piezo actuator connected to the control unit, whereby the second clamping device is configured to switch between a second coupling state for run-inhibiting securing of the carriage to the guide rail against unintended axial movements and a second decoupling state for run-free de-securing of the carriage from the guide rail.

20. The piezoelectric drive unit according to claim 19, wherein the control unit is configured to:
provide the second decoupling state, respectively, temporally precedingly or simultaneously to the first coupling state in order to perform a carriage advance of the carriage according to the longitudinal stroke of the push rod movable from the axial starting position; and/or
provide the second coupling state, respectively, temporally directly subsequently or simultaneously to the performed carriage advance.

21. The piezoelectric drive unit according to claim 16, wherein the longitudinal stroke device and/or the first clamping device and/or the second clamping device is or, respectively, are formed as a respective solid body joint-supported mechanism, especially is or, respectively, are formed durable with respect to an oscillation of at least one piezo actuator from the at least one first, second and/or third piezo actuator.

22. The piezoelectric drive unit according to claim 21, wherein, where the first clamping device is formed as a solid body joint-supported mechanism, the first clamping device comprises a bending spring arranged in a push rod clamping section between the push rod and the carriage, whereby the bending spring is rotatably supported on the drive frame in a bending spring pivot point to form a bending spring lever arm extending toward the push rod clamping section.

23. The piezoelectric drive unit according to claim 21, wherein, where the second clamping device is formed as a solid body joint-supported mechanism, the second clamping device comprises a pivot arm-like clamping lever, whereby the clamping lever is rotatably supported about the longitudinal axis of the linear guide to cause the second coupling state with piezoelectrically actuated expansion of the at least one second piezo actuator and/or third piezo actuator by a deflection force.

24. The piezoelectric drive unit according to claim 21, wherein, where the longitudinal stroke device is formed as a solid body joint-supported mechanism, the longitudinal stroke device comprises a double-sided lever rocker element, whereby the lever rocker element is pivotally supported about a lever rocker rotation axis orthogonal to the longitudinal direction in its central lever rocker midsection at a rear end of the drive frame with respect to a direction of the carriage advance, and for this purpose forms on both sides at the opposite ends a short lever rocker arm and a long lever rocker arm, respectively, in order to cause the longitudinal stroke of the push rod with piezoelectrically actuated expansion of the at least one first piezo actuator by a first piezo stroke.

25. A method of dispensing and/or dosing of medical liquids from a medical container, the method comprising the steps of:
providing a piezoelectric drive unit according to claim 16;
coupling of the carriage to the push rod by the first clamping device of the carriage into a piezoelectrically actuated first coupling state;
generating of a carriage advance of the carriage to an advance position incremental in longitudinal direction, coupled with a longitudinal stroke of the push rod from an axial starting position with acting of the longitudinal stroke device piezoelectrically actuated by means of the at least one first piezo actuator;
decoupling of the carriage from the push rod by means of the first clamping device of the carriage into a piezoelectrically actuated first decoupling state; and
separate motion of the push rod as a retracting motion of the push rod from the advance position incremental in the longitudinal direction back to the starting position, with acting of the longitudinal stroke device piezoelectrically actuated by the at least one first piezo actuator.

26. The method according to claim 25, further comprising the step of controlling oscillation of at least one piezo actuator from the at least one, second and/or third piezo actuator.

27. A medical-technical syringe pump comprising a piezoelectric drive unit according to claim 16.

28. A computer program on a data medium configured for performing the method according to claim 25.
